(19) United States
(12) Patent Application Publication
(10) Pub. No.: US 2006/0270042 A1
(43) Pub. Date: Nov. 30, 2006

(54) High-titer retroviral packaging cells

(76) Inventors: Manuel Caruso, Quebec (CA); Vincent Roy, Quebec (CA); Marie-Christine Caron, Quebec (CA); Karim Ghani, Quebec (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/431,824

(22) Filed: May 11, 2006

Related U.S. Application Data
(63) Continuation of application No. PCT/CA04/01954, filed on Nov. 12, 2004.
(60) Provisional application No. 60/518,651, filed on Nov. 12, 2003.

(51) Int. Cl. C12N 15/867; C12N 5/08; C07K 5/06

(52) U.S. Cl. 435/456; 435/366; 435/325

(57) ABSTRACT

The present invention relates to non-replicative recombinant retrovirus packaging cells able to grow in suspension in a serum-free medium. In particular, the present invention relates to a human embryonic 293SF-based cell line stably expressing gag and pol gene products from the murine Moloney leukemia virus (MLV) and the feline RD114 env gene. This particular combination allows the production of high titer of non-replicative retrovirus pseudotyped and prevents the recombination of plasmids. The recombinant retroviruses produced from these cells are safer and easier to produce for clinical use in gene therapy.

### BACKGROUND OF THE INVENTION

a) Field of the Invention

The present invention relates to the production of a cell line for the packaging of non-replicative retrovirus particles. The present invention also relates to packaging cells capable of growing in a synthetic medium and in suspension to minimize biohazard risks and increase the titers of vision production.

b) Description of Prior Art

The life cycle of retrovirus involves an obligatory stage in which the virus genetic material is inserted into the genome of a host cell by transposition-like events. This step is essential because the inserted viral nucleic acid, the provirus, is replicated through the host cell machinery.

Because retroviruses have genomes of diploid single-stranded RNA (ssRNA), those must be replicated through a double-stranded DNA intermediate prior insertion. The initial conversion of the viral RNA molecule into a double-stranded DNA (dsDNA) molecule is performed by a reverse-transcriptase. The double-stranded DNA is then led to the nucleus, where one or more copies are integrated to the genome by an integrase as proviruses, to further be replicated by the host machinery. The reverse transcriptase and the integrase required for the conversion of ssRNA into dsDNA and for the integration are carried with the viral particle during host cell infection. The proviral DNA is finally transcribed using the host machinery into multiple RNA copies. These RNA molecules will further be translated into virus peptides or integrated into virion particles which will bugg off from the cell and be relieved in the supernatant.

A retrovirus RNA molecule comprises 6 typical regions leading to the expression of multiple proteins by processing reactions. These typical retrovirus region comprise die gag, pol and env gene sequences associated to a psi (Ψ) signal and flanked by 5′ and 3′ long terminal repeats (LTR) sequences. The gag gene leads to the expression of the protein components of the nucleoprotein core of the virion while the pol gene products are implicated in the synthesis of nucleic acid and recombination. The env gene codes for the components of the envelope of retrovirus particle. 5′ and 3′ LTR sequences ensure the correct transcription of the virus RNA into DNA and subsequent integration of the expression vector gene into the chromosomal DNA of the cell. Finally, the psi signal refers to the retroviral packaging signal that controls the efficient packaging of the RNA into the virus particle and therefore, lead to gene transfer.

Because of their ability to form proviruses, retroviruses appeared as adequate tools to modify the genome of particular cells for uses such as gene therapy. Gene therapy using retroviral vectors is generally performed by adding an exogenous nucleic acid sequence to the retroviral RNA vector, packaging this vector into a virion particle and infecting a target host cell. The target cell will then incorporate the exogenous gene as a part of provirus simultaneously to the viral genome.

For safety reasons, retroviral vectors must be replication incompetent, since the target cell would suffer from an infection. Use of vector systems now allows the production of recombinant retroviruses that are unable to replicate by themselves. To avoid replication of retrovirus within target cells, gene therapy makes use of virions that comprise an exogenous gene of interest, which is flanked by the minimal sequences required for retrotranscription into DNA, insertion into host cell genome and proper expression of this gene. The RNA molecule carried by the non-replicative retroviral particle is devoid of gag, pol and env genes and therefore, the target-cell does not produce the nucleoprotein core nor the envelope essential to the replication of the virion.

To infect target cells, encapsidation of the exogenous RNA molecule however remains a necessary step. Therefore, a complementation system must be provided in packaging cells. The virus assembly is generally performed in packaging cells infected with helper virus or stably transfected with constructs at comprise psi-negative gag, pol and env genes.

Transfection of a psi-positive construct that comprises the exogenous gene into these packaging cells lead to the encapsidation of the RNA molecule into a virion. The packaging cells than release retroviral vector particles into the supernatant. As gag/pol and env genes are not carried by the virus particle, they cannot be transferred from packaging cells to target cells.

Packaging cells are frequently designed to es retroviral vectors that are derived from the Moloney murine leukemia virus (MLV). In fact, MLV-derived vectors are the most commonly used vectors in clinical trials for gene therapy. Although these replication-defective recombinant retroviruses can be produced by transient cotransfection of an expression vector comprising the exogenous gene and packaging plasmids that code for gag-pol and env viral proteins, the absence of toxicity of MLV proteins has made possible the generation of stable retrovirus-producer cell lines which are convenient for use in large-scale vector production. To improve the infectious properties of recombinant retroviruses, env gene products of other viruses can be expressed at their surface. This process, known as pseudotyping, is commonly used to modify the virus tropism to make it more infectious and/or more specific to specific cell types. As example, retroviral vectors pseudotyped with the feline RD114 env glycoprotein have been shown to be very promising for gene therapy since they resist to complement inactivation and are efficient to transfer genes into human lymphocytes and hematopoietic stem cells.

One major safety concerned with stable packaging cell lines is to ensure that expression vectors generated from these packaging cells are not contaminated with replication-competent retroviruses (RCRs). RCRs result from the recombination between the expression vector and the packaging plasmids and with some endogenous retroviral elements. It has also been shown that nonhuman primates can develop lymphomas after being grated with genetically modified hematopoietic stem cells contaminated with RCR. To prevent deleterious recombinations between the expression vector and the packaging plasmids, the latest versions of packaging cells use the expression vector and the packaging plasmids that have reduced homologies, rendering almost impossible the generation of replication competent retroviruses.

Although the latest generation of packaging cell lines are used in the protection against RCR, the scale up for clinical uses is imitative since these cell are adherent. Indeed, because the growing environment of cells is limited to the bottom part of a recipient, the production of large retrovirus volumes is cumbersome and therefore, quite expensive. In addition, most of the cells actually used for packaging require the use of animal serum for their growth. This represents another drawback for the existing packaging cell lines since it can lead to contamination by biohazards. Finally, for the production of MLV particles, murine cells are used and produce a characteristic epitope structure at the surface of the virion. This epitope is recognized by the immune system of an organism and causes an antibody-mediated inactivation of the recombinant retrovirus particles.

Recently, a human embryonic kidney (HEK) cell line, the 293SF cell line, has been developed (U.S. Pat. No. 6,210,922) and was shown to bypass the above-mentioned drawbacks generally associated with packaging cells. First, these cells are human cells and therefore do not produce the carbohydrate structure α-galactosyl epitope found at the surface of the virus produced from murine cells and that are, at least in part, responsible for antibody-mediated virus inactivation. In addition, the genomic DNA from these cells do not hybridized with MLV specific probes at low or high stringency, therefore precluding the generation of RCR by recombination with endogenous retrovirus, as found with murine packaging cell lines. Stable packaging cells have already been derived from HEK 293 cells and it seems that they have the property to produce recombinant retroviruses with relatively high titers. Finally, 293SF cells grown in suspension with synthetic media are already available and they are used for the large scale production of proteins and adenoviral vector.

Considering the state of the prior at, it would be desirable to be provided with a packaging cell line that grows in suspension with synthetic medium and is capable of expressing high titers of recombinant retrovirus particles precluding the generation of RCR.

### SUMMARY OF THE INVENTION

One aim of the present invention is to provide a retrovirus packaging cell line, preferably a inn embryonic kidney (HEK) 293-based cell line for the production of a non-replicative retrovirus particle. Particularly, this cell line is capable of growing in suspension, in a serum-free or simultaneously both in suspension and serum free conditions. The nucleic acid component of the non-replicative retrovirus particle comprises a nucleic acid sequence of interest and is capable of being incorporated into the genome of a target host cell.

In one aim of the present invention, the nucleic acid component of the non-replicative retrovirus particle is a ribonucleic acid (RNA) molecule.

Another aim of the present invention is to provide a HEK 293-based cell packaging cell line that comprises HEK 293SF cell line, and particularly the HEK 293SF cell line having the deposit name GP18 (Accession number 190803-01), GPA11 (Accession number 190803-02), and GPRD5 (Accession number 190803-03) athe International Depositary Authority of Canada.

In a further aim of the present invention, there is provided a HEK 293-base packaging cell line that stably expresses the components required for the assembly of the non-replicative retrovirus particle, wherein the components required for the assembly of the non-replicative retrovirus particle are gag, pol and env gene products;

Another aim of the present invention is to provide env, gag and pol gene products that are dissociated from psi (Ψ) factor to prevent their assembly within the non-replicative retrovirus particle.

A further aim of the present invention is to provide gag and pol gene products comprising murine Moloney leukemia virus (MMLV) gene products while the env gene product comprises a feline RD114 env gene product.

In another aim of the present invention, the nucleic acid sequence of interest comprises a gene, a promoter or a combination thereof, and is aimed to be used for gene therapy.

In a further aim of the present invention, the nucleic acid sequence of interest is associated to a psi (Ψ) factor to provide its assembly into the non-replicative retrovirus particle.

There is an aim of the present invention to provided the use of the HEK 293-based packaging cell line to produce non-replicative retrovirus particles for gene therapy. Also is aimed the use of any of the packaging cell lines described herein in the preparation of a composition for ex-vivo gene therapy, or in the production of non replicative retrovirus-particles. The production can be in vitro, in vivo, or ex vivo production.

For the purpose of the present invention the following teems are defined below.

The term “gene product” is intended to mean peptides, a proteins or a RNA molecule encoding these peptides or proteins, resulting from the expression of a gene or any encoding nucleic acid.

The term “IRES” is intended to mean internal ribosomal entry site.

The terms “transfecting” or “transfection” are intended to mean the acquisition of at least one new genetic marker in an eukaryotic cell by the incorporation of exogenous DNA. The erogenous DNA refers to DNA that is not found as a result of cell division or cell multiplication. This DNA may be from a foreign organism or species or from the species from which the a cell originates. In the latter case, transfection can be achieved to over-express a particular gene product.

The term “virion” as used herein is intended to mean the physical virus particle, irrespective of its ability to infect cells or reproduce.

The term “polypeptide” as used herein is intended to mean any protein, peptide, polypeptide or the like that is native or not to the host cell lines. A gene or nucleic acid encoding for the polypeptide may therefore originate from an animal including human, plant, fungal, bacterial or any other living species or organisms. In addition, the polypeptide can be synthesized by a synthetic gene or nucleic acid, synthesized exclusively by human hand or naturally produced and further modified by said human hand, where the polypeptide is different than what is naturally found in the host cell. A recombinant polypeptide may therefore originate from the host cell itself (homologous polypeptide) although having been genetically manipulated for production purposes.

The term “cell line” as used herein refers to cultured cells that can be passaged more than once. The invention relates to cell lines that can be passaged more than 2, to 200 times, or preferably more than any integer between 2 and 200, each number not having been explicitly set forth in the interest of conciseness.

### BRIEF DESCRIPTION OF THE DRAWINGS

FIG. 1: illustrates the structure of bicistronic vectors used for the generation of GP18, GPA11 and GPRD5;

FIG. 2: shows the reverse transcriptase(RT) activity measured in the supernatant from GP clones;

FIG. 3: illustrates the luciferase activity from HT-1080 cells infected with 1 ml of pNC-Luc virus produced transiently from GPRD clones;

FIG. 4: illustrates the luciferase activity from RT-1080 cells infected with 1 ml of pNC-Luc virus produced transiently from GPA clones; and

FIGS. 5A and 5B: illustrate the fluorescence intensity from 143B infected with 1 ml of GFP3 virus produced from GPA11 (A) and GPRD5 (1);

### DETAILED DESCRIPTION OF THE PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided a retrovirus packaging cell line, which can be a human embryonic kidney (HEK) 293-based cell line, for the production of a non-replicative retrovirus particle. The cell line of the present invention stably expresses the components, as for example gag, pol and env gene products, required for the assembly of the non-replicative recombinant retrovirus particle. Moreover, the nucleic acid component of the recombinant retrovirus particles comprises a nucleic acid sequence of interest and is capable of being incorporated into the genome of a target host cell.

In a further embodiment of the present invention, 293-based cells are 293SF cells and preferably the 293SF cells having the deposit name GP18 (Accession number 190803-01), GPA11 (Accession number 190803-02), or GPRD5 (Accession number 190803-03) athe International Depositary Authority of Canada.

In one embodiment of the present invention, the packaging cell line is capable to grow in suspension or in a serum-free medium. The packaging cell line can also be grown both in serum-free medium and suspension simultaneously. Although serum-free medium and the capacity to grow in suspension are the preferred conditions of the present invention to optimize or maximize the yields of retrovirus non-replicative particle titers, the 293SF cells of the present invention can be cultured in an adherent manner with regular serum containing medium to achieve particular purposes. As example, these more conventional eukaryote cell-culture techniques could be used to facilitate transfection of cells or to select clones of cells.

The type of packaging cells used to generate the retrovirus determines the tropism or host range for the virus. These cell lines have the essential retroviral genes—gag, pol and env integrated separately in its genome. Development of synthetic packaging lines is herein a major development that advances utility retrovirus as a gene delivery vehicle. It ends use of replication competent or helper viruses.

In another embodiment of the present invention, the nucleic acid component of the non-replicative retrovirus particle is ribonucleic acid (RNA). This RNA molecule comprises a nucleic acid sequence of interest used for gene therapy. As the psi (Ψ) factor compounds the encapsidation of a RNA molecule into a retrovirus particle, in one embodiment of the present invention, a nucleic acid sequence of interest is associated with a psi factor.

In another embodiment of the present invention, the nucleic acid sequence of interest is a gene, a promoter or a combination thereof. The gene of interest can code for an antibody, an antigen, an antisense RNA, a cytokine, a drug resistance, a hormone, a deficiency, a marker, a trans-dominant, an oncogene regulator, a receptor, a ribozyme, a suicide gene, a tumor suppressor, any other gene, multiple genes or combinations thereof to confer a particular physiological reaction. For the purpose of the present invention, promoter sequences are preferably aimed to provide adequate regulation of the expression of genes under their control. Therefore, the nucleic acid sequence of interest of the present invention preferably comprises a gene located downstream a promoter sequence that controls its expression properly. Both gene and promoter sequences can be naturally occurring nucleic sequences cloned into the viral vector, naturally occurring sequences that have been genetically altered or sync sequences.

The components required for the assembly of said recombinant retrovirus particle are gag, pot and env gene products. Since the psi (Ψ) factor commands the incorporation of a RNA molecule into a virion, it is an embodiment of the present invention to provide env, gag and pot gene products that are dissociated from psi (Ψ) factor. The mRNA stably expressed by the packaging cell line can therefore be translated properly by the cell machinery while avoiding the possibility of being incorporated into the retrovirus particle. Since the gene products of these three genes are essential to the replicative capacity of retroviruses, the absence of the gag, pot and env genes within the nucleic acid sequence carried by the viruses of the present invention confers to the virion the important characteristic of being non-replicative.

In another embodiment of the present invention, the gag and pot gene products can be derived from a murine Moloney leukemia virus (MLV) and the env gene product is preferably, but not limited to, derived from the feline env RD114 virus. A MLV gag-pol expression plasmid containing a histidinol resistance selectable marker (hisD) linked to an internal ribosomal entry site (IRES) is preferably used to generate gag-pol expressing 293SF clones. The env expression plasmid is preferably constructed by cloning a feline RD114 env gene followed by a puromycin-N-acetyl transferase (puro^{r}) gene, in a commercial plasmid. Both vectors can generate one transcript the IRES allows the initiation of the hisD gene in a cap-independent manner, and in the other vector, the puro^{r} gene will be translated by reinitiation of translation.

Use of MLV gag and pol and RD114 env genes is preferred because one of the main safety concern packaging cell line stably expressing the component required for the assembly of non-replicative retroviruses is to ensure that the exogenous nucleic acid molecules cannot recombinate to form replicative-competent retroviruses. Since the expression vector of the present invention and the packaging plasmids have reduced homologies, the generation of replication competent-retroviruses is almost impossible.

In addition, the feline RD114 env gene is preferred since it confers to the vision resistance to complement inactivation and efficiency of genes transfer into human lymphocytes and hematopoietic stem cells. Although this combination of gag, pol and env gene is preferred, a skilled artisan could use any vector and plasmid sets capable of providing similar properties to the packaging cell line.

The HEK 293-based packaging cell line is used to produce high titer of non-replicative retroviral particles for that use. Gene therapy as intended herein consists in the modification of the genome of at least one cell of a patient. The modification of the genome comprises the addition of genetic material into a cell genetic pool by gene insertion either to correct a heredity disease or to modify the characteristics of the cell via expression of the newly inserted gene. Therefore, gene therapy can be used in a healthy patient to genetically enhance a particular physical trait or physiological characteristic. However, the purpose of the present invention is preferably the cure of diseases that include, but are not limited to, cancer, monogenic diseases, infectious diseases, vascular diseases and otter diseases.

Other genetic diseases that can be treated according to the present invention include, but are not limited to, Chronic Granulomatous Disease, Duchenne Muscular Dystrophy, Retinobastoma, Cystic Fibrosis, Wilms Tumor, Neurofibromatosis Type 1, Testis Determining Factor, Choroideremia Fragile X Syndrome, Familial Polyposis Coli, Kallmann Syndrome, Aniridia, Myotonic Dystrophy, Lowe Syndrome, Norrie Syndrome, Menkes Disease, X-Linked Agammaglobulinemia, Glycerol Kinase Deficiency, Adrenoleukodystrophy, Neurofibromatosis Type 2, Huntington Disease, Von Hippel-Lindau Disease, Spinocerebellar Ataxia 1, Lissencephaly, Wilson Disease, Tuberous Sclerosis, McLeod Syndrome, Polycystic Kidney Disease Type 1, Dentatorubral Pallidoluysian Atrophy, Fragile X “E”, Achondroplasia Wiskott Aldrich Syndrome, Early Onset Breast/Ovarian Cancer (BRCA1), Diastrophic Dysplasia, Aarskog-Scott Syndrome, Spinocerebellar Ataxia 3, Congenital Adrenal Hypoplasia, Emery-Dreifuss Muscular Dystrophy, Machado-Joseph Disease, Spinal Muscular Atrophy, Chondrodysplasia Punctata, Limb-Girdle Muscular Dystrophy, Ocular Albinism, Ataxia Telangiectasia, Alzheimer's Disease, (Chromosome 1 and/or 14), Hypophosphatemic Rickets, Hereditary Multiple Exostoses (EXT1 and/or EXT2), Bloom Syndrome, Early Onset Breast Cancer (BRCA2), Friedreich's Ataxia, Progressive Myoclonic Epilepsy, Treacher Coins Syndrome, Long QT Syndrome (Chromosome 11), Barth Syndrome, Simpson-Golabi-Behmel Syndrome, Werner's Syndrome, X-Linked Retinitis Pigmentosa (RP3), Polycystic Kidney Disease, Type 2, Basal Cell Nevus Syndrome, X-linked Myotubular Myopathy, Anhidrotic Ectodermal Dysplasia, Hemochromatosis, Chediak-Higashi Syndrome, Fanconi Anemia A, Hermansky-Pudlak Syndrome, Spinocerebellar Ataxia 2, CADASIL (Hereditary Stroke), Rieger Syndrome, Maturity-Onset Diabetes of the Young (Chromosome 12), Holt-Oram Syndrome, Angelman Syndrome, Juvenile Glaucoma, Stargardt's Disease, Multiple Endocrine Neoplasia Type 1, Niemann-Pick Disease Type C, Alagille Syndrome, Familial Mediterranean Fever, Tuberous Sclerosis 1, Dystonia, Spinocerebellar Ataxia Type 7, Optz Syndrome, Situs Inversus, Deafness (DFNA1), Pendred Syndrome, APECED (Inherited Autoimmunity)

Infectious diseases include acquired immunodeficiency syndrome (AIDS) while other diseases comprise, but are not limited to, coronary artery disease, amyotrophic lateral sclerosis (ALS) and rheumatoid arthritis.

The packaging cells of the present invention can be also used to produce non-replicative retroviruses for any other purposes. These purposes include, but are not limited to, gene marking to assess the feasibility of human somatic transgenesis, the fate of a bone marrow autograft in cancer patients, the fate of bone marrow and peripheral blood cell autografts, and the risk of reimplanting malignant cells.

Route of administration of the non-replicative retrovirus particles to the patient can be performed by bone implant, bone marrow transplantation, intravenous, intraarterial, intraarticular, intrabronchial, intracoronary, intracranial, sublingual, intradermal, intramuscular, subcutaneous, intrahepatic, intramyocardial, intranasal, intrabronchial, intrathecal, intraprostatic, intraperitoneal, intrapleural, intravesical, intratumoral intraulcer or skin patch administration. The source of target cell for the gene therapy can be autologous, allogenic, syngeneic or xenogeneic cells.

In another embodiment, there is provided the use of a packaging cell line as defined hereinabove to produce a recombinant retrovirus for use in gene therapy.

The present invention will be more readily understood by referring to the following examples which are given to illustrate the invention rather than to limit its scope.

### EXAMPLE I

### Construction of the pCI-IRDpuro^{r} Plasmid

The RD114 env expression plasmid is constructed as follows: a 2003 bp HindIII/ApaI env fragment from a RD114 infectious virus clone, SC3C, is treated with the T4 DNA polymerase to blunt both extremities of the DNA fragment as currently known in the art. The blunted fragment is then cloned in the SmaI restriction site of the polylinker site of a pCI vector (Promega), to generate a pCI-RD114 plasmid (pCI-RD). This plasmid is further used to construct a pCI-RD plasmid comprising a selection marker. For the purpose of the present invention a gene encoding for a resistance to puromycin was chosen (puro^{r}). Puromycin is an aminonucleoside antibiotic produce by Streptomyces alboninger that inhibits in eukaryotic, as well as prokaryotic cells. The puro^{r} gene encodes a puromycin N-acetyl-transferase (PAC) that confers resistance to mammalian cells. The pCI-RD plasmid comprising the puro^{r} gene (pCI-RDpuro^{r}) was constructed is follow: a 670 bp puro^{r} gene digested by PvuII/EcoRV and blunted using T4 DNA polymerase. The puro^{r} was then cloned, as known in the art, into a NotI restriction site of the pCI-RD plasmid, the stop codon of the env gene and the ATG of the Puro^{r} gene being separated by 140 nucleotides.

### EXAMPLE II

### Obtention of a 293SF Cell Line Stably Expressing Murine GAG and POL Genes and Feline RD114 ENV Gene

Material and Methods

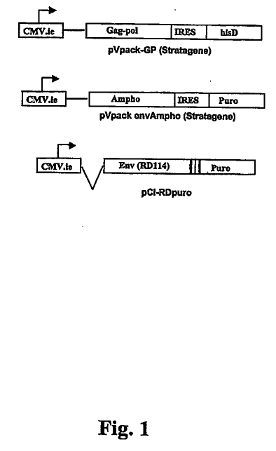

293SF cells were cultured in Dulbecco's modified Eagle's medium transfected and maintained in a medium complemented with 10% fetal calf serum and antibiotics. 293SF cells were then transfected with the pVPack-GP vector (Stratagene) by the calcium phosphate procedure. This vector comprises gag and pol genes from the Moloney murine leukemia virus. To obtain the clones that stably expressed both genes, cells were selected with histidinol (250 mM) for two weeks. The pVPack-GP (FIG. 1) plasmid includes a histidinol resistance gene, histidinol dehydrogenase (hisD), that allows cells having incorporated the vector into their genome to survive histidinol treatment.

The histidinol-resistant clones were isolated and an analysis of the expression of gag and pol genes was assessed by measuring the expression level of reverse transcriptase (RT). The presence of RT in the supernatant of GP clones was measured as follows: 5 μl of supernatant was added to 25 μl of a RT master mix containing 20 μCi/ml dTT³²P, 50 mM Tris-Cl, 75 mM KCl, 2 mM DTT, 1 mM MnCl₂, 5 μl/ml poly(rA)+oligo dT and 0.5% (v/v) NP-40. The reactions were incubated 4 hours at 37° C. in a 96-well plate, and 6 μl of the total volume was spotted on a DE81 filter paper. The filter was then washed 5 times with 1×SSC for 5 mites end twice with 85% ethanol for five minutes. The radioactivity associated to the filter was then revealed and quantified with a phosphorimager.

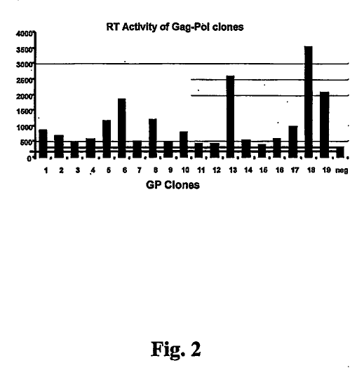

The clone the expressed the highest RT levels (293SF-GP18 clone) was selected to perform a further stable transfection with the pCI-RDpuro^{r} plaid. To obtain 293SF-GP cells stably expressing the RD114 env gone, cells were incubated in the presence of 0.2 μg/ml of puromycin for two weeks. Twelve (12) puromycin-resistant clones (GPRD clones) were obtained and then harvested for further characterization. RT activity of each clone is shown in FIG. 2.

### EXAMPLE III

### Expression of an Exogenous Gene in GRPD Cell Line

One day prior to transient transfection, 3×10⁵ cells from each GPRD clones were plated in 6-well plate. Each clone was individually transfected by the calcium phosphate procedure with 6 μl of pNC-Luc. The pNC-Luc retroviral plasmid used to screen GPRD clones is derived from a Moloney murine leukemia vector which has a neomycin resistance gene (Neo), under the control of an internal CMV promoter.

The next day, 1 ml of each transfected clone was harvested and used to infect HT-1080 cells in the presence of 8 μg/ml polybrene. The target cells had been plated the day before at a density of 3×10⁵ cells per well in 6-well plates. A luciferase assay was performed one day after infection of HT-1080 cells, and also an GPRD clones at the time of the supernatant harvest to normalize the transfection efficiency. Cells were trypsinized, washed twice with PBS, resuspended in 0.25 M Tris-HCl, pH 8.0 and cell extracts were obtained by freezing/thawing 3 times. Cell debris were pelleted at 14,000 g for 2 minutes, and supernatants were used for enzymatic assays.

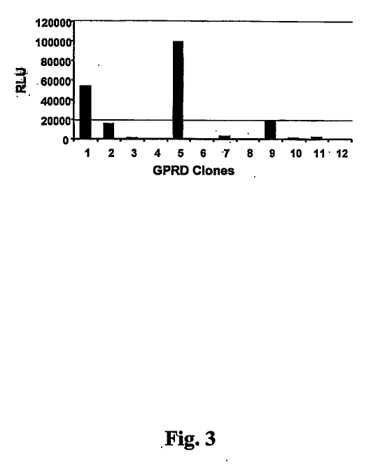

Luciferase assays were performed as follows: 30 μl of cell extract were added into 350 μl of reaction buffer (25 mM glycylglycine, pH 7.8, 10 mM MgSO₄, 2 mM adenosine triphosphate). For each reaction, 100 μl of luciferin was added by injection and light emission was measured for 2 seconds us a Lumat LB 9507 luminometer (EG & G Berthold, Bad Wildbad, Germany). GPRD clones were individually transfected with the pNC-Lac vector. Two days later, the supernatant was collected and used to infect HT-1080 cells. Two more days later, luciferase activity was measured and detected in infected cells with 9 out of 12 supernatants (FIG. 3). The luciferase activity in HT-1080 cells was the highest with the was from clone 5 which was then selected for further characterization as a packaging cell line

### EXAMPLE IV

### Production of Luciferase in Transformed Clones GPA11 and GPRD5

Material and Methods:

Plasmids

The RD114 env expression plasmids were constructed as follows: 2003 bp HindIII/ApaI env fragment from SC3C, a RD114 infectious virus clone (gift from S. O'Brien), was treated by the T4 DNA polymerase and cloned in SmaI in pCI (Promega) to generate pCI-RD. Next, pCI-RDpuro was constructed by inserting a 670 bp Puro^{r} gene that was digested by PvuII/EcoRV and cloned in PCI-RD in NotI that was previously blunted by the T4 DNA polymerase. The distance between the stop codon of the env gene and the ATG of the Puro^{r} gene is 140 nucleotides.

The pNC-Luc retroviral plasmid used to screen GPRD and GPA clones is derived from a Moloney murine leukemia vector which has a neomycin resistance gene (Neo^{r}) undo the control of an internal CMV i.e. promoter.

GFP3 is a bicistronic retroviral plasmid which contain a mutant of the herpes simplex virus thymidine kinase (TK30) and GFP under the control of the LTR. GFP is located downstream an IRES and it is then expressed in a cap-independent manner.

Cell Culture, Stable Transfection and Retroviral Infection

293SF, HT-1080 and 143B cells were cultured in Dulbecco's modified Eagle's medium. All these cell lines were maintained in medium complemented with 10% fetal calf serum and antibiotics. GP clones were generated by transfection of 293SF with the pVPack-GP vector (Stratagene) by the calcium phosphate procedure. Clones were selected in media depleted in histidine and with histidinol at 0.250 mM for two weeks and they were isolated and analyzed for the presence of RT in their supernatant. The GP18 clone (the clone that release the highest level of RT) was transfected with pCI-RDpuro to generate GPRD clones and with pVpack envAmpho (Stratagene) to generate GPA clones. Isolated clones were selected for two weeks with 0.2 μg/ml of puromycin and they were harvested for further characterization.

Recombinant retroviruses were generated by 6 infections by spinoculation (of GPRD5 and GPA11 with GFP3 virus produced from PG13 packaging cells, a GFP retroviral plasmid. GPRD5 and GPA11 were more than 90% positive for GFP expression.

Viral infectivity was evaluated on 143B cells. Briefly, 143B cells were seeded at 3×10⁵ cells/well in a 6-well plate, and hey were infected the next day with 1 ml of viral supernatant in the presence of 8 μg/ml PB. Forty-eight hours later, cells were trypsinized and analyzed for GFP fluorescence.

RT Assay

The presence of RT in the supernatant of GP clones was measured as follows: 5 μl of supernatant was added to 25 μl of a RT master mix containing 20 μCi/ml dTT³²P, 50 mM Tis-Cl, 75 mM KCl, 2 mM DTT, 1 mM MnCl₂, 5 μg/ml poly(rA)+oligo dT and 0.5% (v/v) NP-40. The reactions were incubated 4 hours at 37° C. in a 96-well plate, and 6 μl of the total volume was spotted on a DE81 filter paper. The filter was then washed 5 times with 1×SSC for 5 minutes and twice with 85% ethanol for five minutes. The radioactivity associated to the filter was then revealed and quantified with a phosphorimager.

Screening of Producer Cell Lines

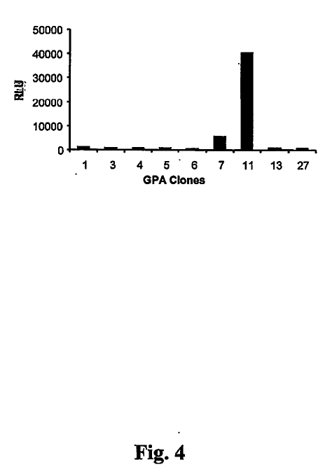

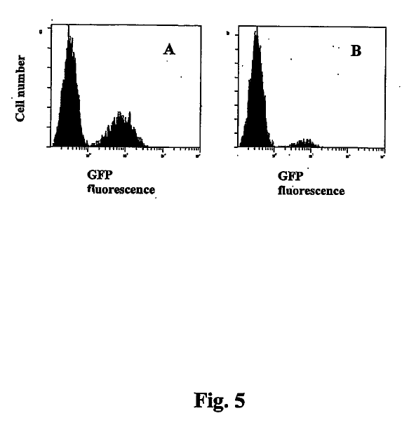

One day prior to transient transfection, 3×10⁵ cells from each GPRD or GPA clones were plated in 6-well plates. Each clone was individually transfected by the calcium phosphate procedure with 6 μg of pNC-Luc. The next day, 1 ml of each transfected clone was harvested and used to infect HT-1080 cells in the presence of 8 μg/ml polybrene. The target cells had been plated the day before at a density of 3×10⁵ cells per well in 6-well plates. The luciferase assay was performed one day after infection of HT-1080 cells, and also on GPRD clones at the time of the supernatant harvest to normalize the transfection efficiency (FIG. 4). Cells were trypsinized, washed twice with PBS, resuspended in 0.25 M Tris-HCl, pH 8.0 and cell extracts were obtained by freezing/thawing 3 times. Cell debris were pelleted at 14 000 g for 2 minutes, and supernatants were used for enzymatic assays. Luciferase assay was performed as follows: 30 μl of cell exact were added into 350 μl of reaction buffer (25 mM glycylglycine, pH 7, 8, 10 mM MgSO₄, 2 mM adenosine triphosphate). For each reaction, 100 μl of luciferin was added by injection and light emission was measured for 2 seconds using Lumat LB 9507 huminometer (BG& G Berthold, Bad Wildbad, Germany) (FIG. 5).

While the invention has been described in connection with specific embodiments thereof, it will be understood that it is capable of further modifications and this application is intended to cover any variations, uses, or adaptations of the invention following, in general, the principles of the invention and including such departs from the present disclosure as come within known or customary practice within the art to which the invention pertains and as may be applied to the essential features hereinbefore set forth, and as follows in the scope of the appended claims.

1. A retrovirus packaging HEK cell line for the production of non-replicative retrovirus particles, said cell line being genetically transformed for stably expressing components required for assembly of non-replicative retrovirus particles capable of incorporation into the genome of a host cell and comprised of a nucleic acid encoding polypeptide of interest, wherein said retrovirus packaging cell line is cultured in suspension in a serum-free medium.

2. The retrovirus packaging HEK cell line of claim 1 being selected from the group consisting of cell line GP18 (Accession number 190803-01 filed on Aug. 19, 2003), GPA11 (Accession number 190803-02 filed on Aug. 19, 2003) and GPRD5 (Accession number 190803-03 filed on Aug. 19, 2003) at the International Depositary Authority of Canada.

3. The retrovirus packaging HEK cell line of claim 1 being a human embryonic kidney cell line.

4. The retrovirus packaging HEK cell line of claim 1, wherein said packaging cell line is composed of 293 cells.

5. The retrovirus packaging HEK cell line of claim 1, wherein said components required for the assembly of said non-replicative retrovirus particle are expressed by a nucleic acid sequence comprising gag, pol and env genes.

6. The retrovirus packaging HEK cell line of claim 5, wherein said env, gag and pol gene products are dissociated from psi (v) factor to avoid assembly of RNA encoding said gene products in said non-replicative retrovirus particle.

7. The retrovirus packaging HEK cell line of claim 5, wherein said gag and pol gene products originate from murine Moloney leukemia virus.

8. The retrovirus packaging HEK cell line of claim 5, wherein said env gene product originates from feline RD114 env gene.

9. The retrovirus packaging HEK cell line of claim 1, wherein said nucleic acid sequence is composed of RNA.

10. The retrovirus packaging HEK cell line of claim 1, wherein said nucleic acid sequence of interest comprises a gene, a promoter or a combination thereof.

11. The retrovirus packaging HEK cell line of claim 1, wherein said nucleic acid is associated to a psi (Ψ) factor to allow its encapsidation into said non-replicative retrovirus particles.

12. A method for producing non-replicative retrovirus particles comprising maintaining HEK cells genetically transformed with replication defective retrovirus particles in suspension in a serum free culture medium in condition for allowing replication of said cells.

11-15. (canceled)

16. The method of claim 12, wherein the production is in vitro or in vivo.

17. A method for preparing a composition for ex vivo gene therapy comprising maintaining the retrovirus packaging HEK cell line of claim 1 in a serum free culture medium in condition for allowing replication of said cells.
